(19) United States
(12) Patent Application Publication
(10) Pub. No.: US 2026/0263228 A1
(43) Pub. Date: Sep. 10, 2026

(54) ARTHROPLASTY FEMORAL IMPLANT DEVICES

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Roger Ryan Dees, JR., Drummonds, TN (US); Forrest A. James, Memphis, TN (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/167,639

(22) PCT Filed: Jun. 18, 2024
(86) PCT No.: PCT/US2024/034447

§ 371 Date: Sep. 22, 2025

Related U.S. Application Data
(60) Provisional application No. 63/522,359, filed on Jun. 21, 2023.

(51) Int. Cl. A61F 2/38

(52) U.S. Cl. CPC A61F 2/3859; A61F 2/389

(57) ABSTRACT

Disclosed herein is an angled-wall femoral component (500) of a cam-post knee arthroplasty system for a posterior cruciate ligament (PCL)-substituting total knee arthroplasty (TKA) or revision TKA (rTKA) procedure, such as a cruciate substituting (CS), a bicruciate substituting (BCS), or posterior stabilized (PS) procedure. The angled-wall femoral component may include a femoral box (502) configured to receive the tibial post (110) of a corresponding tibial component (100). An anterior wall (508) of the femoral box is anteriorly angled and may include an anterior cam (561). The anterior wall is angled anteriorly to allow for clearance between an anterior side of the tibial post and the anterior wall, while allowing for operative engagement between the tibial post and the anterior cam during flexion and extension of the knee implanted with the femoral component.

### CROSS-REFERENCE TO RELATED APPLICATION

This application claims the benefit of U.S. Provisional Patent Application Ser. No. 63/522,359, filed Jun. 21, 2023, and titled “Arthroplasty Femoral Implant Devices,” the entire contents of which application are hereby incorporated by reference in their entirety.

### TECHNICAL FIELD

The present disclosure is directed to orthopedic implants and, more specifically, to a femoral component configured for a cam-post (or cam-and-post) mechanism used in a posterior cruciate ligament (PCL)-substituting knee arthroplasty procedure, for example, a cruciate substituting (CS), a bicruciate substituting (BCS), or posterior stabilized (PS) femoral component.

### BACKGROUND

The biomechanics of the knee are highly complex and influenced by multiple anatomical characteristics. Significant disease or injury affecting the knee joint may be treated by a knee arthroplasty procedure to surgically replace the ends, or a portion thereof, of the femur and/or tibia with a prosthetic knee device that may include a prosthetic femoral implant and/or a tibial implant.

Knee arthroplasty procedures can be grouped into different classification types based on how the cruciate ligaments are managed. For example, posterior cruciate ligament (PCL)-substituting procedures may include a cruciate substituting (CS), a bicruciate substituting (BCS), or a posterior stabilized (PS) total knee arthroplasty (TKA) procedure (or revision TKA (rTKA)) that involve removal of the posterior cruciate ligament (PCL) (and potentially the anterior cruciate ligament (ACL)) of the patient's knee while substituting for the PCL by way of a cam-post (or cam-and-post) mechanism which helps to control anterior translation during knee flexion.

A PCL-substituting tibial component includes an insert having a post extending superiorly from the tibia that engages with a femoral box of the femoral component. The femoral box may include an anterior cam and/or a posterior cam that interfaces with and stabilizes the implant, for instance, against tibial sliding when the knee is flexed and/or other unwanted implant movement.

The design of a femoral box must strike a balance between supporting the operation of the tibial post (e.g., providing an articulation surface and cam mechanism) and constraining unwanted motion, such as varus and valgus motion during flexion. Conventional cam-post arthroplasty systems do not achieve normal knee kinematics or motion and generally have a more limited range of motion than a normal knee due to interference between the femoral box walls and the post. For example, conventional designs typically provide a limited space between the insert post and the femoral box walls to increase insert post strength and contact area between a femoral posterior cam and the insert post. However, the limited space between the insert post and the femoral box walls impedes the ability of the post-and-cam mechanism to emulate the natural motion of a healthy knee.

Accordingly, knee arthroplasty patients would benefit from cam-post mechanism implant components that support natural operation of the knee, including facilitating desired knee kinematics, to alleviate the pain and instability associated with conventional prosthetic knee devices, while also providing sufficient strength and stability to avoid implant failure.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

The present disclosure describes an angled-wall femoral component of a prosthetic knee implant system. In any preceding or subsequent example, an angled-wall femoral component may be used in a cam-post (or cam-and-post) knee implant system for a posterior cruciate ligament (PCL)-substituting total knee arthroplasty (TKA) or revision TKA (rTKA) procedure.

In any preceding or subsequent example, a PCL-substituting implant system may include an angled-wall femoral component configured to be installed on a prepared distal end of a femur and a tibial component configured to be installed on a prepared proximal end of a tibia.

In any preceding or subsequent example, the PCL-substituting implant system may be configured for a cruciate substituting (CS) procedure, a bicruciate substituting (BCS) procedure, or posterior stabilized (PS) procedure. In any preceding or subsequent example, the PCL-substituting implant system may be configured for an rTKA procedure.

In any preceding or subsequent example, the tibial component may include a tibial post extending superiorly from a superior surface of the tibial component. In any preceding or subsequent example, the tibial post may be formed extending from a tibial insert coupled to the tibial component.

In any preceding or subsequent example, the angled-wall femoral component may include a femoral box configured to receive the tibial post. In any preceding or subsequent example, the femoral box may be open on an inferior side facing the tibial component. In any preceding or subsequent example, the femoral box may be enclosed on a superior side opposite the inferior side.

In any preceding or subsequent example, the femoral box may include a posterior side having a posterior cam. In any preceding or subsequent example, the femoral box may include an anterior side having an anterior cam.

In any preceding or subsequent example, an anterior wall or anterior side wall of the femoral box may be angled. In any preceding or subsequent example, the anterior wall may include at least one angled section. In any preceding or subsequent example, the anterior wall may be concave and/or stepped.

In any preceding or subsequent example, the anterior wall may be angled to allow for clearance between an anterior side of the tibial post and the anterior wall, while allowing for engagement between the tibial post and the anterior cam. In any preceding or subsequent example, the anterior wall may be angled anteriorly (e.g., away from a posterior side of the femoral box).

In any preceding or subsequent example, the anterior wall may be angled with respect to a non-angled anterior wall extending from the anterior cam to a superior end (or top) of the femoral box. For instance, the anterior wall may be angled anteriorly (e.g., away from a posterior side of the femoral box) at an angle relative to a line extending horizontally through the arc or radius of the anterior cam.

In any preceding or subsequent example, the angle of the anterior wall may be about 1 degree, about 2 degrees, about 3 degrees, about 4 degrees, about 5 degrees, about 6 degrees, about 7 degrees, about 8 degrees, about 9 degrees, about 10 degrees, about 12 degrees, about 14 degrees, about 15 degrees, about 16 degrees, about 18 degrees, about 20 degrees, or about 25 degrees and any value or range between any two of these values (including endpoints). In any preceding or subsequent example, the angle may be about 7 degrees. In any preceding or subsequent example, the angle may be about 7 degrees to about 10 degrees. In any preceding or subsequent example, the angle may be about 5 degrees to about 10 degrees. In any preceding or subsequent example, the angle may be about 10 degrees.

In any preceding or subsequent example, the angle of the anterior wall may be any angle sufficient to provide clearance between the anterior wall and a tibial post of a corresponding tibial component. In any preceding or subsequent example, the angle of the anterior wall may be any angle sufficient to provide clearance between the anterior wall and a tibial post of a corresponding tibial component, while allowing for engagement between the tibial post and an anterior cam.

In any preceding or subsequent example, the tibial post may have a post height, post width, and a post angle (i.e., an angle of an anterior side of the tibial post). In any preceding or subsequent example, the angle of the anterior wall may be determined, at least in part, based on the post height, the post width, and/or the post angle. The angles of an anterior wall according to any preceding or subsequent example may be based on a non-angled tibial post (e.g., 90 degree or substantially 90-degree angle between the tibial post and a base or insert surface of the tibial component). In any preceding or subsequent example, an angle of the tibial post may lead to a corresponding adjustment to the angle of the anterior wall (e.g., a post angled 1 degree anteriorly may lead to an angled wall with an additional 1 degree of angle to maintain no contact between the tibial post and the anterior wall).

In any preceding or subsequent example, the anterior wall may be concave, stepped, or otherwise shaped and/or angled to allow for the tibial post to avoid contact with the anterior wall through various degrees of extension/flexion (“clearance” or “non-contact motion”). In any preceding or subsequent example, the anterior wall may be concave, stepped, or otherwise shaped and/or angled to allow non-contact motion for a tibial post through about −10 degrees to about 30 degrees of extension. In any preceding or subsequent example, the non-contact motion may be from about 0 degrees to about 10 degrees of extension, from about 0 degrees to about 15 degrees of extension, from about 0 degrees to about 20 degrees of extension, from about 0 degrees to about 25 degrees of extension, from about 0 degrees to about 30 degrees of extension, from about 0 degrees to about 35 degrees of extension, from about 0 degrees to about 40 degrees of extension, and any value or range between any two of these values (including endpoints).

In any preceding or subsequent example, the anterior wall may be concave, stepped, or otherwise shaped and/or angled to allow for non-contact motion while the tibial post is engaged with the anterior cam through a radius or arc of the anterior cam of about 1 mm to about 20 mm and/or through about 0 degrees to about 30 degrees of extension.

In any preceding or subsequent example, a femoral component of a knee implant system may include a femoral box configured to receive a tibial post of a corresponding tibial component, the femoral box having a posterior side wall and an anterior side wall arranged opposite the posterior side wall, wherein the anterior wall is formed with an angled section angled anteriorly to allow for clearance between an anterior side of the tibial post and the anterior side wall during extension and flexion of a knee implanted with the knee implant system.

In any preceding or subsequent example of the femoral component, the femoral component further includes an anterior cam arranged on the anterior side wall and configured to engage the anterior side of the tibial post during extension of the knee, wherein the anterior wall is formed with the angled section to allow, during extension and flexion of the knee, for clearance between the anterior side of the tibial post and the anterior side wall, and engagement between the anterior side of the tibial post and the anterior cam.

In any preceding or subsequent example of the femoral component, the anterior cam is arranged on an inferior end of the anterior side wall.

In any preceding or subsequent example of the femoral component, the angled portion extends superiorly from the anterior cam to a superior end of the femoral box.

In any preceding or subsequent example of the femoral component, the anterior side wall is concave or stepped.

In any preceding or subsequent example of the femoral component, the angled section is angled anteriorly by an angle sufficient to provide clearance between the tibial post and the anterior side wall during extension and flexion of the knee.

In any preceding or subsequent example of the femoral component, the angled section is angled anteriorly by an angle sufficient to provide clearance between the tibial post and the anterior side wall during about 0 degrees to about 30 degrees of extension.

In any preceding or subsequent example of the femoral component, the angled section is angled anteriorly by about 5 degrees to about 10 degrees.

In any preceding or subsequent example of the femoral component, the angled section is angled anteriorly by about 10 degrees.

In any preceding or subsequent example of the femoral component, the angled section is angled anteriorly by about 7 degrees.

In any preceding or subsequent example of the femoral component, the angled section is angled anteriorly by an angle determined based on at least one of a dimension or a post angle of the tibial post.

In any preceding or subsequent example of the femoral component, a length of the angled section is about 5 mm to about 20 mm.

In any preceding or subsequent example of the femoral component, the femoral component is configured as one of a cruciate substituting (CS) femoral component, a bicruciate substituting (BCS) femoral component, or a posterior stabilized (PS) femoral component.

In any preceding or subsequent example, a knee implant system may include a tibial component including a base and a tibial post having an anterior side, the tibial post extending superiorly from the base; and a femoral component configured to engage the base of the tibial component, the femoral component including a femoral box configured to receive the tibial post, the femoral box having a posterior side wall and an anterior side wall arranged opposite the posterior side wall, wherein the anterior side wall is formed with an angled section angled anteriorly to allow for clearance between the anterior side of the tibial post and the anterior side wall during extension and flexion of a knee implanted with the knee implant system.

In any preceding or subsequent example of the knee implant system, the femoral component further includes an anterior cam arranged on the anterior side wall and configured to engage the anterior side of the tibial post during extension of the knee, the anterior side wall is formed with the angled section to allow, during extension and flexion of the knee, for clearance between the anterior side of the tibial post and the anterior side wall, and engagement between the anterior side of the tibial post and the anterior cam.

In any preceding or subsequent example of the knee implant system, the anterior cam is arranged on an inferior end of the anterior side wall.

In any preceding or subsequent example of the knee implant system, the angled portion extends superiorly from the anterior cam to a superior end of the femoral box.

In any preceding or subsequent example of the knee implant system, the anterior side wall is concave or stepped.

In any preceding or subsequent example of the knee implant system, the angled section is angled anteriorly by an angle sufficient to provide clearance between the tibial post and the anterior side wall during extension and flexion of the knee.

In any preceding or subsequent example of the knee implant system, the angled section is angled anteriorly by an angle sufficient to provide clearance between the tibial post and the anterior side wall during about 0 degrees to about 30 degrees of extension.

In any preceding or subsequent example of the knee implant system, the angled section is angled anteriorly by about 5 degrees to about 10 degrees.

In any preceding or subsequent example of the knee implant system, the angled section is angled anteriorly by about 10 degrees.

In any preceding or subsequent example of the knee implant system, the angled section is angled anteriorly by about 7 degrees.

In any preceding or subsequent example of the knee implant system, the angled section is angled anteriorly by an angle determined based on at least one of a dimension or a post angle of the tibial post.

In any preceding or subsequent example of the knee implant system, a length of the angled section is about 5 mm to about 20 mm.

In any preceding or subsequent example of the knee implant system, the knee implant system is configured as one of a cruciate substituting (CS) knee implant system, a bicruciate substituting (BCS) knee implant system, or a posterior stabilized (PS) knee implant system.

In any preceding or subsequent example, a method of performing knee arthroplasty on a patient may include implanting a tibial component including on a prepared proximal end of a tibia of a knee joint of the patient, the tibial component including a base and a tibial post having an anterior side, the tibial post extending superiorly from the base, and implanting a femoral component on a prepared distal end of a femur of the knee, the femoral component configured to engage the base of the tibial component and including a femoral box configured to receive the tibial post, the femoral box having a posterior side wall and an anterior side wall arranged opposite the posterior side wall, wherein the anterior side wall is formed with an angled section angled anteriorly to allow for clearance between the anterior side of the tibial post and the anterior side wall during extension and flexion of a knee implanted with the knee implant system.

In any preceding or subsequent example of the method, the femoral component further includes an anterior cam arranged on the anterior side wall and configured to engage the anterior side of the tibial post during extension of the knee, the anterior side wall is formed with the angled section to allow, during extension and flexion of the knee, for clearance between the anterior side of the tibial post and the anterior side wall, and engagement between the anterior side of the tibial post and the anterior cam.

In any preceding or subsequent example of the method, the anterior cam is arranged on an inferior end of the anterior side wall.

In any preceding or subsequent example of the method, the angled portion extends superiorly from the anterior cam to a superior end of the femoral box.

In any preceding or subsequent example of the method, the anterior side wall is concave or stepped.

In any preceding or subsequent example of the method, the angled section is angled anteriorly by an angle sufficient to provide clearance between the tibial post and the anterior side wall during extension and flexion of the knee.

In any preceding or subsequent example of the method, the angled section is angled anteriorly by an angle sufficient to provide clearance between the tibial post and the anterior side wall during about 0 degrees to about 30 degrees of extension.

In any preceding or subsequent example of the method, the angled section is angled anteriorly by about 5 degrees to about 10 degrees.

In any preceding or subsequent example of the method, the angled section is angled anteriorly by about 10 degrees.

In any preceding or subsequent example of the method, the angled section is angled anteriorly by about 7 degrees.

In any preceding or subsequent example of the method, the angled section is angled anteriorly by an angle determined based on at least one of a dimension or a post angle of the tibial post.

In any preceding or subsequent example of the method, a length of the angled section is about 5 mm to about 20 mm.

In any preceding or subsequent example of the method, the knee arthroplasty includes one of the following procedures: cruciate substituting (CS), bicruciate substituting (BCS), or posterior stabilized (PS).

Examples of the present disclosure provide numerous technological features and advantages over conventional systems. In one non-limiting example, an angled-wall femoral component may reduce, minimize, or even eliminate interference of an anterior wall (or anterior side wall) of a femoral box (including, in particular, an enclosed femoral box with a walled top or superior end). In an additional non-limiting example, an angled-wall femoral component may allow for increased tibial post articulation with an anterior cam while avoiding unwanted contact between the anterior side of the tibial post and the anterior wall.

Accordingly, as described in more detail in the present disclosure, some examples may provide a femoral component formed with a geometry configured to provide improved implant stability and kinematic performance throughout the complete range of motion of the affected knee, when compared with conventional implant systems.

Examples are not limited in this context. Additional technological advantages and technical features over conventional systems would be known to those of skill in the art based on the present disclosure.

Further features and advantages of at least some examples of the present disclosure, as well as the structure and operation of various examples of the present disclosure, are described in detail below with reference to the accompanying drawings.

### BRIEF DESCRIPTION OF THE DRAWINGS

By way of example, features of the disclosed components and systems will now be described, with reference to the accompanying drawings, in which:

FIGS. 1A-1C depict an illustrative example of a cam-post knee implant system in accordance with the present disclosure;

FIGS. 2A-2C depict an internal sagittal view of an illustrative example of an angled-wall femoral component of a cam-post knee implant system in accordance with the present disclosure;

FIGS. 3A-3C depict an internal sagittal view of an illustrative example of an angled-wall femoral component of a cam-post knee implant system in comparison to femoral components with non-angled anterior walls in accordance with the present disclosure; and

FIGS. 4A-4D depict an internal sagittal view of an illustrative example of movement of a cam-post knee implant system in accordance with the present disclosure.

It should be understood that the drawings are not necessarily to scale and that the disclosed examples are sometimes illustrated diagrammatically and in partial views. In certain instances, details which are not necessary for an understanding of the disclosed methods and devices, or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

Various features of an improved femoral component of a knee implant system will now be described more fully hereinafter with reference to the accompanying drawings, in which one or more features of the femoral component will be shown and described. It should be appreciated that the various features may be used independently of, or in combination, with each other. It will be appreciated that a femoral component as disclosed herein may be embodied in many different forms and should not be construed as being limited to the examples set forth herein. Rather, these examples are provided so that this disclosure will convey certain features of the femoral component to those skilled in the art.

The present disclosure describes an angled-wall femoral component for a cam-post (or cam-and-post) knee implant system. In general, cam-post implant systems are used in a posterior cruciate ligament (PCL)-substituting total knee arthroplasty (TKA) or revision TKA (rTKA) procedure in which the PCL (and typically the anterior cruciate ligament (ACL)) has been removed. Cam-post knee implant systems employ a tibial post and a femoral cam mechanism to guide anteroposterior knee motion and rollback in lieu of the posterior cruciate ligament (PCL).

Non-limiting examples of a PCL-substituting procedure may include a bicruciate stabilized (BCS) or posterior-stabilized (PS) TKA or rTKA procedure. Although BCS and PS implant systems are used in the present disclosure to illustrate various features of the femoral component, examples are not so limited, as the femoral component may be used with any type of knee arthroplasty procedure and/or implant system that may operate or may be configured to operate according to the described examples.

In various examples, the angled-wall femoral component may include an anterior wall (or anterior side wall) of a femoral box that includes an anterior cam and an angled anterior wall. In some examples, the angled anterior wall may be configured to allow for clearance for a tibial post within the femoral box during knee flexion/extension to avoid contact between the post and the anterior wall while allowing for engagement between the post and the anterior cam, for instance, to facilitate natural kinematics of a knee using the knee implant system.

Accordingly, as described in more detail in the present disclosure, some examples may provide a femoral component formed with a geometry configured to provide improved implant stability and kinematic performance throughout the complete range of motion of the affected knee, including, within flexion, mid-flexion, and extension regions, when compared with conventional implant systems.

As will be described herein, an implant device, including a femoral component thereof, may have various shapes, sizes, and/or configurations of other physical properties. It should be appreciated that the implant device may be provided in any suitable shape and/or configuration, which, as will be appreciated by one of ordinary skill in the art, may be dependent on the location and type of patient bone being fixed. For example, an implant device may include various bone conforming segments configured to correspond with different anatomical features and/or prepared portions of patient anatomy. In addition, the femoral component may be a part of an implant device arranged and configured to span, contact, be affixed to, and/or the like various portions of a human knee, including without limitation, the tibia and/or femur.

A femoral component according to some examples may include any now known or hereafter developed additional features. The femoral component may be manufactured from any suitable material now known or hereafter developed, including, for example, metals, polymers, plastics, ceramics, resorbable, non-resorbable, composite materials, and/or the like. Suitable materials may include, without limitation, titanium, stainless steel, cobalt chrome, polyetheretherketone (PEEK), polyethylene, ultra-high molecular weight polyethylene (UHMWPE), resorbable polylactic acid (PLA), polyglycolic acid (PGA), combinations or alloys of such materials or any other appropriate material that has sufficient strength to be secured to and hold bone, while also having sufficient biocompatibility to be implanted into a human body.

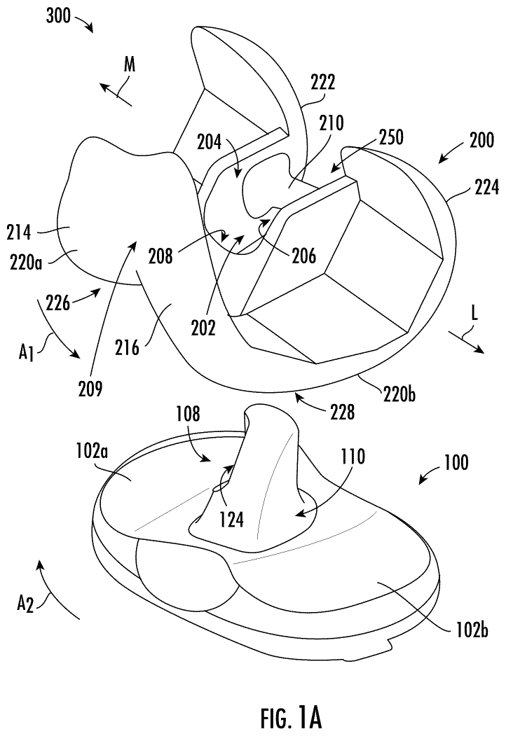

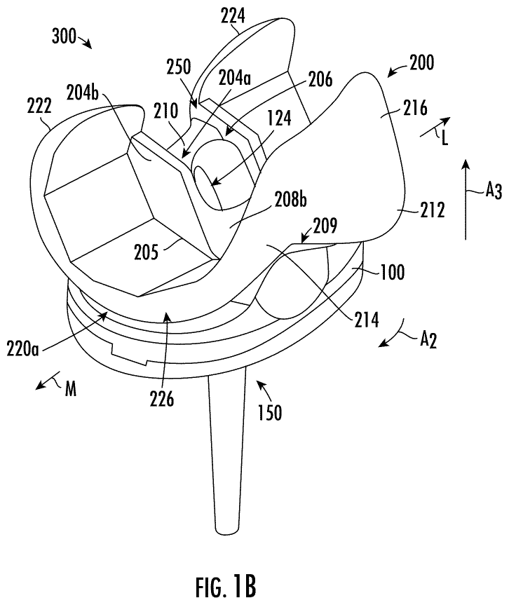

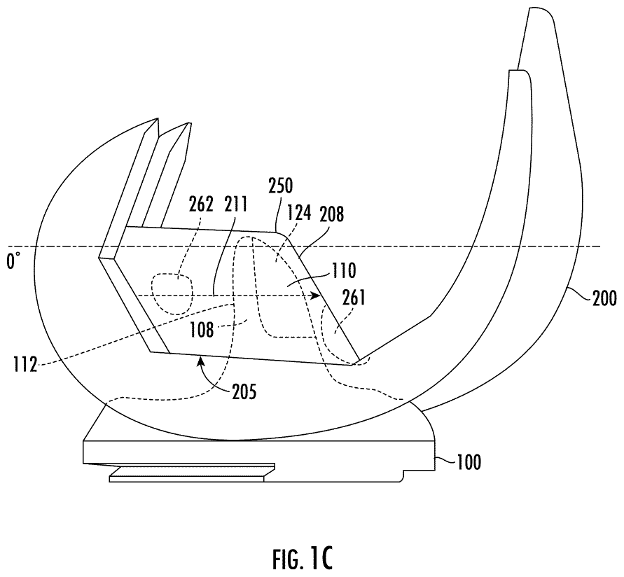

FIGS. 1A-1C depict an illustrative example of a cam-post knee implant system in accordance with the present disclosure. More specifically, FIGS. 1A-1C depict an orthopedic implant system 300 having a tibial component 100 with a femoral component 200. In some examples, femoral component 200 and/or tibial component 100 may be configured as primary TKA implant components. In various examples, femoral component 200 and/or tibial component 100 may be configured as revision TKA implant components (for instance, to accommodate a larger post, for example, due to the stem boss required on a revision femoral component). For instance, for rTKA procedures and implant devices, the control and stability requirements from the prostheses are increased compared with TKA implants as compromises in soft tissue constraints increase.

As shown in FIGS. 1A-1C, tibial component 100 may be coupled with an insert 108. Femoral component 200 has a medial condylar section 214 and a lateral condylar section 216 with a trochlear grove 209 joining medial and lateral condylar sections 214 and 216. Medial and lateral condylar sections 214 and 216 are disposed apart from each other to form an intercondylar recess or notch. Each condylar section 214 and 216 has a distal region 228 and 226, respectively, for engaging a portion of a tibial component 100 or insert 108 when the knee joint is extended and partially flexed and a posterior portion 222 and 224, respectively, for engaging a portion of tibial component 100 or insert 108 when the knee joint is flexed at angles of substantially 90 degrees or greater.

In use, insert 108 fits with the femoral component 200 by inserting a post 110 extending superiorly from insert 108 into a box 202 (which may be referred to, without the intention to limit, as a “femoral box,” a “PS box,” or a “BCS box”) formed within femoral component 200. In some examples, box 202 may be formed by four walls (“box walls”) comprising medial side wall 204, lateral side wall 206, posterior side wall 210 and anterior side wall 208. In the non-limiting example femoral component depicted in FIGS. 1A and 1B, box 202 has an opening on a superior side 250 of box 202. In some examples, box 202 may be completely enclosed, for example, a fifth or superior wall (not shown) may be included on superior side 250 of box.

Referring to FIG. 1C, in various examples, anterior side wall 208 may include an anterior cam 261. In some examples, posterior side wall 210 may include or may be formed as a posterior cam 262. When femoral component 200 and insert 108 are coupled, post 110 is received within an opening between anterior cam 261 and posterior cam 262.

Femoral component 200 and/or insert 108, and/or components thereof, such as post 110 and/or box 202, may be the same or similar to and/or have the same or similar dimensions to the JOURNEY II BCS implant and/or LEGION PS implant, and/or components thereof, provided by Smith & Nephew of London, United Kingdom.

In some examples, box 202 may have a width of about 10 mm to about 25 mm.

In various examples, box 202 may have a width of about 16.5 mm. In some examples, box 202 may have a width of about 23 mm. In various examples, box 202 may have a height of about 10 mm to about 40 mm. In some examples, box 202 may have a height of about 16 mm to about 22.8 mm.

In various examples, post 110 may have a height of about 10 mm to about 25 mm. In some examples, post 110 may have a height of about 15 mm to about 20 mm. In various examples, post may have a width of about 2 mm to about 10 mm. In some examples, post may have a width of about 3 mm to about 6 mm. In various examples, post 110 may have a wider width at an inferior portion than at a superior portion.

FIGS. 1B and 1C depict system 300 during full knee extension. As shown in FIG. 1B, recess 124 is located on the medial anterior portion of post 110 and disposed along the inside portion 204a of medial wall 204. Medial wall 204 includes an outer face 204b and a lower border 205. During flexion and extension, femoral component 200 articulates with respect to insert 108 as surfaces 220a and 220b articulate with respect to medial portion 102a and lateral portion 102b of the base, respectively.

In extension of the knee, an anterior surface of post 110 may engage anterior cam 261, for example, to provide anterior stabilization. In some examples, clearance is provided such that anterior cam 261 does not contact the post 110 during normal standing, for example, with the leg straight at or about 0 degrees of flexion.

As the knee flexes and femoral component 200 and insert 108 articulate with respect to each other, they rotate axially relative to each other about a longitudinal axis 106 of post 110. In particular, femoral component 200 rotates along arrow Al and insert 108 articulates axially along arrow A2. As articulation and relative rotation occur, femoral component 200 and insert 108 will separate along the anterior side, with the anterior portion of femoral component 200 rising along the direction of arrow A3. As that occurs, interface 124 rotates axially along the path of arrow A2, and lower border 205 of box 202 rises vertically along arrow A3. During that process, recess 124 allows the anterior portion of post 110 to clear the corner of medial wall 204.

The operation of a cam-post implant system with an anterior cam is challenging because the post may come into contact with an anterior face (e.g., interior surface of anterior wall (or anterior side wall) 208) of the box. The unwanted contact between the post and the anterior wall may occur due to various properties of the post, such as the height, width, and/or placement of the post on the tibial insert. This may be a particular challenge with rTKA procedures due to the configurations of the revision tibial component and corresponding post.

Modifying the femoral box and/or anterior wall, such as removing material from the anterior wall or moving the anterior wall anteriorly may provide unwanted affects, such as cam-post articulation with poor kinematics and/or moving the interference points, but not sufficiently eliminating anterior wall-post interference.

Accordingly, in some examples, an angled-wall femoral component may include an angled anterior wall with an anterior cam. The angled anterior wall may provide clearance for the post to engage with the anterior cam while avoiding unwanted contact with the anterior wall. In various examples, an angled-wall femoral component may provide clearance for the tibial post throughout the range of motion to provide improved kinematics of the post-surgical knee, including natural or substantially natural kinematics, compared with conventional systems.

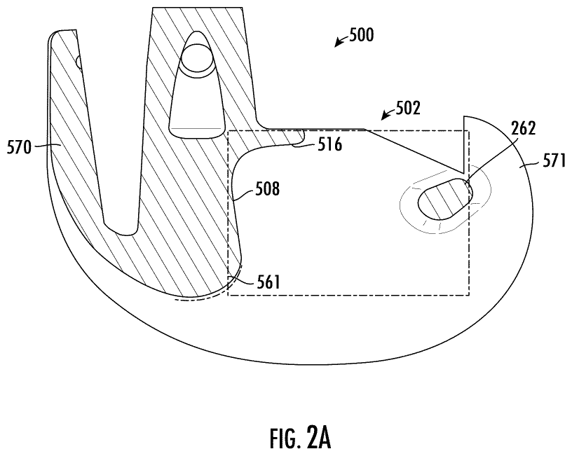

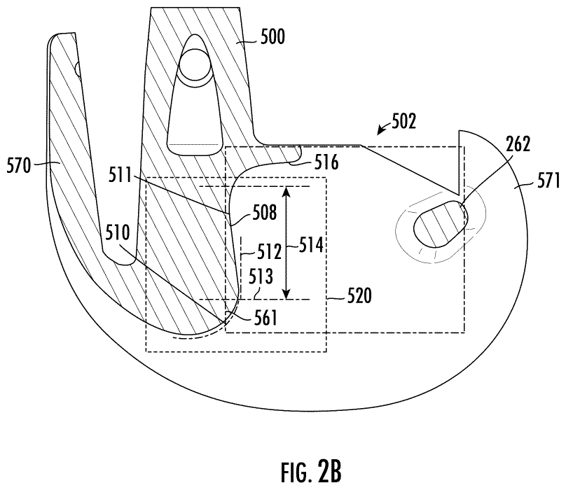

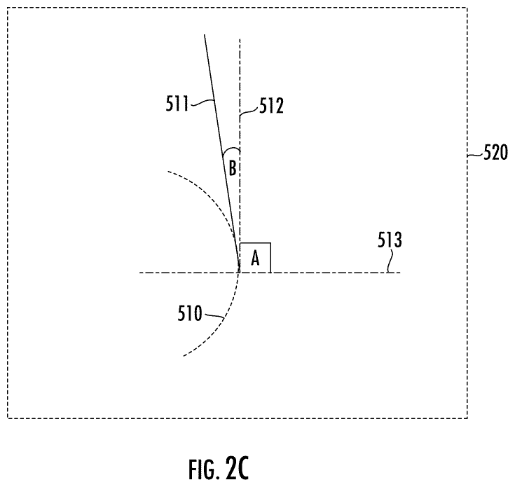

FIGS. 2A-2C depict an internal sagittal view of an illustrative example of an angled-wall femoral component of a cam-post knee implant system in accordance with the present disclosure. As shown in FIG. 2A, an angled-wall femoral component 500 may include an anterior side 570 and a posterior side 571. A box or femoral box 502 may be arranged within femoral component. Box 502 may include one or more cams, such as posterior cam 262 and/or anterior cam 561. In various examples, anterior cam 561 may be arranged on an inferior end of an anterior wall 508 that extends superiorly to a superior wall 516.

Referring to FIGS. 2B and 2C, anterior wall 508 and anterior cam 561 may have various properties, such as geometric or other configuration properties, alone or with respect to each other as indicated in detailed area 520.

In various examples, anterior cam 561 may have a radius 510. In some examples, radius 510 may define a size of anterior cam and/or a distance of engagement between anterior cam 561 and post 110. In various examples, radius 510 may be about 1 mm, about 2 mm, about 3 mm, about 4 mm, about 5 mm, about 6 mm, about 7 mm, about 10 mm, about 15 mm, about 20 mm, and any value or range between any two of these values (including endpoints).

In some examples, anterior wall 508 (or the angled section of anterior wall 508) may have a length 514, for instance, extending from anterior cam 561 (for example, from a center line 513 extending from a center of radius 510) to superior wall 516 (or other superior end of box 502). In various examples, anterior wall 508 (or the angled section of anterior wall 508) may have a length of about 5 mm, about 6 mm, about 7 mm, about 8 mm, about 10 mm, about 12 mm, about 15 mm, about 16 mm, about 18 mm, about 20 mm, about 22 mm, about 25 mm, and any value or range between any two of these values (including endpoints).

In various examples, anterior wall 508 may be an angled wall. In some examples, anterior wall 508 may be angled to allow for clearance between an anterior side of a tibial post (e.g., post 110) and anterior wall 508, while allowing for engagement between the tibial post and anterior cam 561. In various examples, anterior wall 508 may be angled anteriorly (e.g., away from a posterior side of box 502).

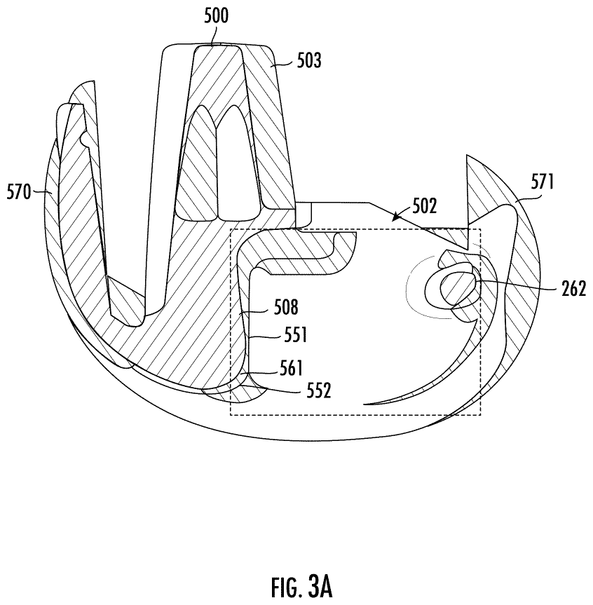

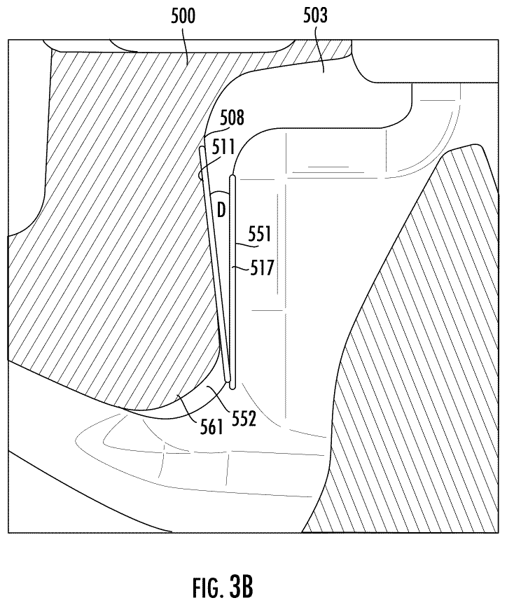

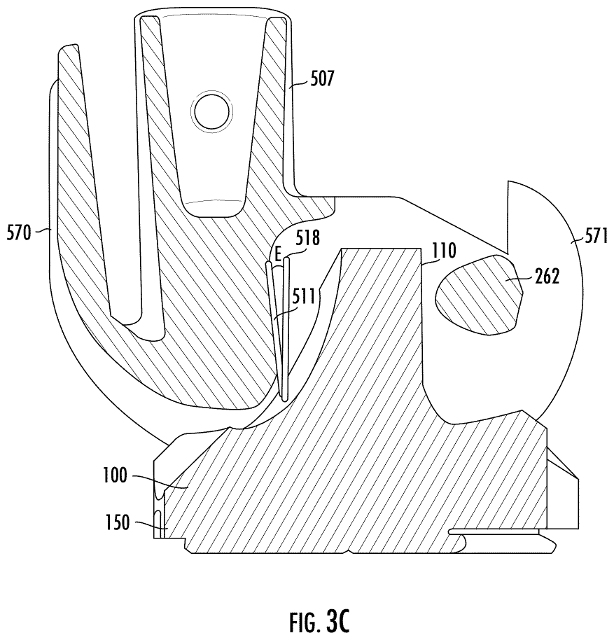

For example, with reference to FIG. 2C, a line 511 extending along at least a portion of anterior wall 508 may be angled by Angle B with respect to a line 512 forming a right 90-degree Angle A with line 513 (i.e., line 512 is perpendicular to line 513), for instance, extending horizontally through anterior cam 561. In various examples, the angle or angle characteristic of anterior wall 508 (e.g., the value of Angle B) may indicate an offset from a non-angled wall (e.g., line 512) (see, for instance, FIGS. 3A-3C). In some examples, at least a portion of anterior wall 508 may be concave, stepped, or otherwise shaped to avoid contact between anterior wall and post 110. In some examples, a portion of anterior wall 508 may not be angled (e.g., a portion of anterior wall 508 where anterior cam 561 is arranged) and an angled portion.

In various examples, Angle B (the angle of anterior wall 508) may be about 1 degree, about 2 degrees, about 3 degrees, about 4 degrees, about 5 degrees, about 6 degrees, about 7 degrees, about 8 degrees, about 9 degrees, about 10 degrees, about 12 degrees, about 14 degrees, about 15 degrees, about 16 degrees, about 18 degrees, about 20 degrees, about 25 degrees, and any value or range between any two of these values (including endpoints). In some examples, the angle may be about 7 degrees. In various examples, Angle B may be about 5 degrees to about 10 degrees. In some examples, Angle B may be about 10 degrees. In various examples, Angle B may be any angle sufficient to provide clearance between anterior wall 508 and a tibial post of a corresponding tibial component during flexion/extension of the knee. In some examples, Angle B may be any angle sufficient to provide clearance between the anterior wall 508 and a tibial post of a corresponding tibial component, while allowing for engagement between the tibial post and anterior cam 561 during flexion/extension of the knee.

In some examples, a tibial post engaging with femoral component 500 may have a post height (e.g., about 20 mm to about 45 mm), post width (e.g., about 10 mm to about 20 mm), and a post angle (i.e., an angle of an anterior side of the tibial post). In various examples, Angle B may be determined, at least in part, based on the post height, the post width, and/or the post angle. Angle B may be based on a non-angled tibial post (e.g., 90 degree or substantially 90-degree angle between the tibial post and a base or insert surface of the tibial component). In some examples, an angle of the tibial post may lead to a corresponding adjustment to Angle B, for instance, to maintain clearance between the tibial post and anterior wall 508 during flexion/extension of the knee.

In some examples, Angle B and/or other anterior wall angles described in the present disclosure may be based on the configuration of a corresponding tibial post having standard dimensions, for instance, a post height of about 34 mm to about 40 mm, a post width of about 16 mm, and a post angle of about 0 degrees. However, Angle B and/or other anterior wall angles described in the present disclosure may be based on tibial posts having different dimensions and/or post angle.

In various examples, anterior wall 508 may be concave, stepped, or otherwise shaped and/or angled to allow for a tibial post to avoid contact with anterior wall 508 through various degrees of extension/flexion (“clearance” or “non-contact motion”). In some examples, anterior wall 508 may be concave, stepped, or otherwise shaped and/or angled to allow non-contact motion for a tibial post through about 0 degrees to about 30 degrees of extension. In various examples, the non-contact motion may be from about 0 degrees to about 10 degrees of extension, from about 0 degrees to about 15 degrees of extension, from about 0 degrees to about 20 degrees of extension, from about 0 degrees to about 25 degrees of extension, from about 0 degrees to about 30 degrees of extension, from about 0 degrees to about 35 degrees of extension, from about 0 degrees to about 40 degrees of extension, and any value or range between any two of these values (including endpoints).

In some examples, anterior wall 508 may be concave, stepped, or otherwise shaped and/or angled to allow for non-contact motion while the tibial post is engaged with anterior cam 561 through a radius or arc of anterior cam 561 of about 1 mm to about 20 mm and/or through about 0 degrees to about 30 degrees of extension.

FIGS. 3A-3C depict an internal sagittal view of an illustrative example of an angled-wall femoral component of a cam-post knee implant system in comparison to femoral components with non-angled anterior walls in accordance with the present disclosure.

Referring to FIG. 3A, angled-wall femoral component 500 is depicted overlaid a non-angled-wall femoral component 503 having an anterior cam 552 and an anterior wall 551. Femoral component 503 may be or may be substantially similar to a femoral component of a LEGION PS femoral component. Referring to FIG. 3B, Angle D indicates the difference between line 511 associated with anterior wall 508 and line 517 associated with anterior wall 551 of non-angled-wall femoral component 503.

Referring to FIG. 3C, therein is depicted a non-angled-wall femoral component 507. Femoral component 507 may be or may be substantially similar to a femoral component of a JOURNEY II BCS femoral component. Angle E indicates the difference between line 511 associated with anterior wall 508 and line 518 associated with an anterior wall of non-angled-wall femoral component 507.

In various examples, Angle D and/or Angle E may be about 1 degree, about 2 degrees, about 3 degrees, about 4 degrees, about 5 degrees, about 6 degrees, about 7 degrees, about 8 degrees, about 9 degrees, about 10 degrees, about 12 degrees, about 14 degrees, about 15 degrees, about 16 degrees, about 18 degrees, about 20 degrees, about 25 degrees, and any value or range between any two of these values (including endpoints).

In various examples, Angle D and/or Angle E may be any angle sufficient to provide clearance between anterior wall 508 and a tibial post of a corresponding tibial component, while allowing for engagement between the tibial post and anterior cam 561.

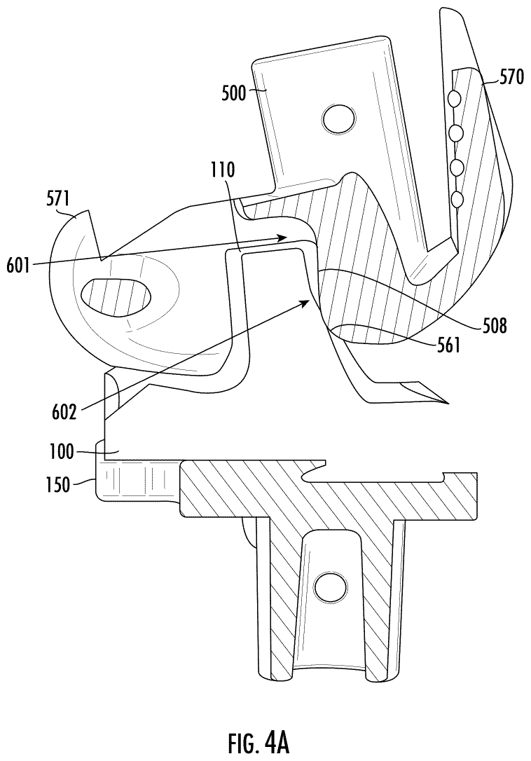

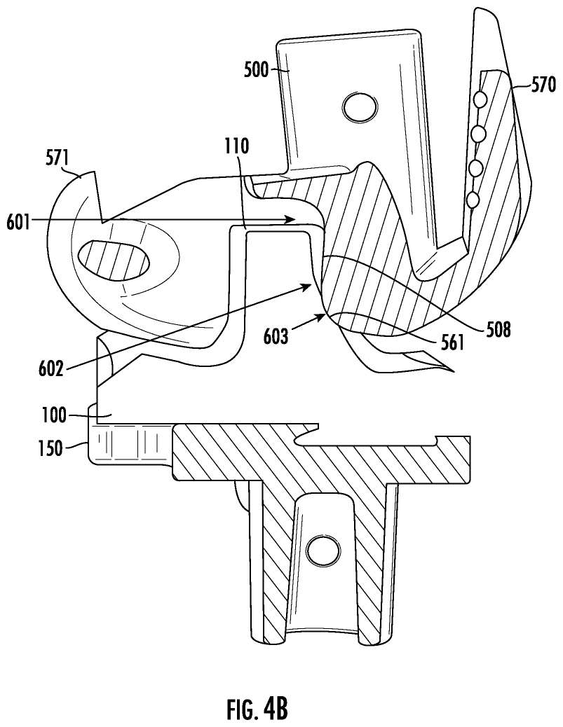

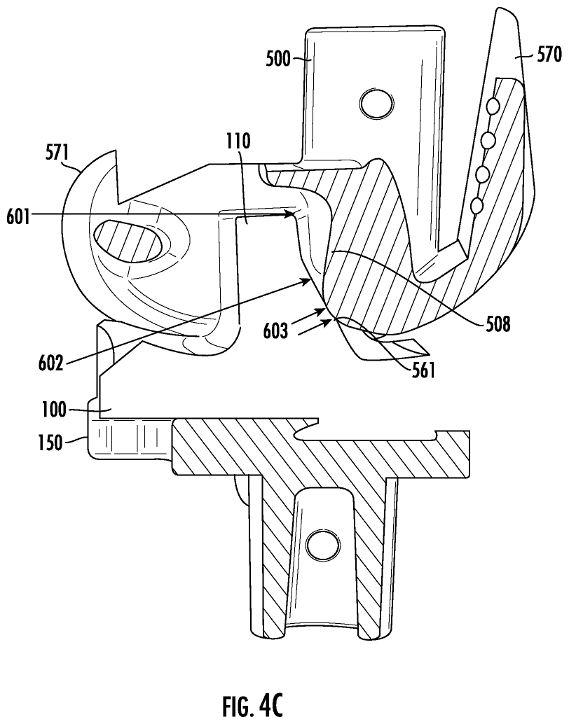

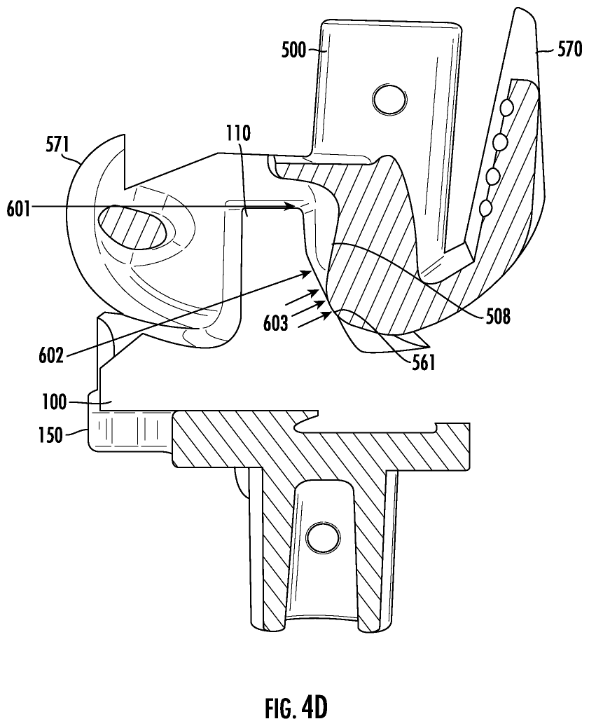

FIGS. 4A-4D depict an internal sagittal view of an illustrative example of movement of a cam-post knee implant system in accordance with the present disclosure. In general, FIGS. 4A-4D depict a cam-post knee implant system having an angled wall femoral component from a first degree of flexion (FIG. 4A) to full or substantially full extension (FIG. 4D). In some examples, FIG. 4A may depict about 10 degrees of flexion/extension, FIG. 4B may depict about 5 degrees of flexion/extension, FIG. 4C may depict about 0 degrees of flexion/extension (e.g., femoral and tibial stems parallel or substantially parallel), and FIG. 4D may depict about −3 degrees of flexion/extension.

Referring to FIG. 4A, therein is depicted angled-wall femoral component 500 in flexion, for example, about 10 degrees of flexion/extension. The angle of anterior wall 508 of angled-wall femoral component 500 may allow for post clearance 601 (for instance, proximal post clearance) for post 110 while supporting cam contact 602 (anterior cam contact) (see also 603 in FIGS. 4B-4D) between post 110 and anterior cam 561. For instance, in FIGS. 4A-4B, post 110 is not contacting anterior wall 508 throughout the range of motion. With a conventional, non-angled-wall femoral box, post 110 may have been contacting anterior wall during the range of motion depicted in one or more of FIGS. 4A-4D, including, for instance, at least FIGS. 4A-4C.

Accordingly, as indicated in FIGS. 4A-4D, increased cam contact 603 may be facilitated between post 110 and anterior cam 561 to support articulation of femoral component 500 with natural or substantially natural kinematics, particularly compared with conventional non-angled-wall femoral components.

The foregoing description has broad application. While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples. Rather these examples should be considered as illustrative and not restrictive in character. All changes and modifications that come within the spirit of the examples are to be considered within the scope of the disclosure. The present disclosure should be given the full scope defined by the language of the following claims, and equivalents thereof. The discussion of any examples is meant only to be explanatory and is not intended to suggest that the scope of the disclosure, including the claims, is limited to these examples. In other words, while illustrative examples of the disclosure have been described in detail herein, it is to be understood that the concepts, features, and/or the like may be otherwise variously embodied and employed, and that the appended claims are intended to be construed to include such variations, except as limited by the prior art. Unless otherwise defined, all technical terms used herein have the same meaning as commonly understood by one of ordinary skill in the art to which the disclosure belongs.

Directional terms such as top, bottom, superior, inferior, medial, lateral, anterior, posterior, proximal, distal, upper, lower, upward, downward, left, right, longitudinal, front, back, above, below, vertical, horizontal, radial, axial, clockwise, and counter-clockwise) and the like may have been used herein. Such directional references are only used for identification purposes to aid the reader's understanding of the present disclosure. For example, the term “distal” may refer to the end farthest away from the medical professional/operator when introducing a device into a patient, while the term “proximal” may refer to the end closest to the medical professional when introducing a device into a patient. Such directional references do not necessarily create limitations, particularly as to the position, orientation, or use of this disclosure. As such, directional references should not be limited to specific coordinate orientations, distances, or sizes, but are used to describe relative positions referencing particular examples. Such terms are not generally limiting to the scope of the claims made herein. Any example or feature of any section, portion, or any other component shown or particularly described in relation to various examples of similar sections, portions, or components herein may be interchangeably applied to any other similar examples or features shown or described herein.

It should be understood that, as described herein, an “example” (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated examples are to be understood as examples (unless otherwise stated), and other manners of embodying the described concepts or features, such as may be understood by one of ordinary skill in the art upon learning the concepts or features from the present disclosure, are within the scope of the disclosure. Furthermore, references to “one example” of the present disclosure are not intended to be interpreted as excluding the existence of additional examples that also incorporate the recited features.

In addition, it will be appreciated that while the Figures may show one or more examples of concepts or features together in a single example of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one example can be used separately, or with another example to yield a still further example. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited. It will be further understood that the terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

The phrases “at least one”, “one or more”, and “and/or”, as used herein, are open-ended expressions that are both conjunctive and disjunctive in operation. The terms “a” (or “an”), “one or more” and “at least one” can be used interchangeably herein.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples or configurations. Moreover, the following claims are hereby incorporated into this Detailed Description by this reference, with each claim standing on its own as a separate example of the present disclosure.

1. A femoral component of a knee implant system, comprising:
a femoral box configured to receive a tibial post of a corresponding tibial component, the femoral box having a posterior side wall and an anterior side wall arranged opposite the posterior side wall,
wherein the anterior side wall is formed with an angled section angled anteriorly to allow for clearance between an anterior side of the tibial post and the anterior side wall during extension and flexion of a knee implanted with the knee implant system.

2. The femoral component of the knee implant system of claim 1, further comprising:
an anterior cam arranged on the anterior side wall and configured to engage the anterior side of the tibial post during extension of the knee,
wherein the anterior side wall is formed with the angled section to allow, during extension and flexion of the knee, for:
clearance between the anterior side of the tibial post and the anterior side wall, and
engagement between the anterior side of the tibial post and the anterior cam.

3. The femoral component of the knee implant system of claim 2, wherein the anterior cam is arranged on an inferior end of the anterior side wall.

4. The femoral component of the knee implant system of claim 2, wherein the angled portion extends superiorly from the anterior cam to a superior end of the femoral box.

5. The femoral component of the knee implant system of claim 1, wherein the anterior side wall is concave or stepped.

6. The femoral component of the knee implant system of claim 1, wherein the angled section is angled anteriorly by an angle sufficient to provide clearance between the tibial post and the anterior side wall during extension and flexion of the knee.

7. The femoral component of the knee implant system of claim 1, wherein the angled section is angled anteriorly by an angle sufficient to provide clearance between the tibial post and the anterior side wall during about 0 degrees to about 30 degrees of extension.

8. The femoral component of the knee implant system of claim 1, wherein the angled section is angled anteriorly by about 5 degrees to about 10 degrees.

9-13. (canceled)

14. A knee implant system, comprising:
a tibial component comprising a base and a tibial post having an anterior side, the tibial post extending superiorly from the base; and
a femoral component configured to engage the base of the tibial component, the femoral component comprising:
a femoral box configured to receive the tibial post, the femoral box having a posterior side wall and an anterior side wall arranged opposite the posterior side wall,
wherein the anterior side wall is formed with an angled section angled anteriorly to allow for clearance between the anterior side of the tibial post and the anterior side wall during extension and flexion of a knee implanted with the knee implant system.

15. The knee implant system of claim 14, the femoral component further comprising:
an anterior cam arranged on the anterior side wall and configured to engage the anterior side of the tibial post during extension of the knee,
wherein the anterior side wall is formed with the angled section to allow, during extension and flexion of the knee, for:
clearance between the anterior side of the tibial post and the anterior side wall, and
engagement between the anterior side of the tibial post and the anterior cam.

16. The knee implant system of claim 15, wherein the anterior cam is arranged on an inferior end of the anterior side wall.

17. The knee implant system of claim 15, wherein the angled portion extends superiorly from the anterior cam to a superior end of the femoral box.

18. The knee implant system of claim 14, wherein the anterior side wall is concave or stepped.

19. The knee implant system of claim 14, wherein the angled section is angled anteriorly by an angle sufficient to provide clearance between the tibial post and the anterior side wall during extension and flexion of the knee.

20. The knee implant system of claim 14, wherein the angled section is angled anteriorly by an angle sufficient to provide clearance between the tibial post and the anterior side wall during about 0 degrees to about 30 degrees of extension.

21. The knee implant system of claim 14, wherein the angled section is angled anteriorly by about 5 degrees to about 10 degrees.

22. The knee implant system of claim 14, wherein the angled section is angled anteriorly by about 10 degrees.

23. The knee implant system of claim 14, wherein the angled section is angled anteriorly by about 7 degrees.

24. The knee implant system of claim 14, wherein the angled section is angled anteriorly by an angle determined based on at least one of a dimension or a post angle of the tibial post.

25. The knee implant system of claim 14, wherein a length of the angled section is about 5 mm to about 20 mm.

26. The knee implant system of claim 14, wherein the knee implant system is configured as one of a cruciate substituting (CS) knee implant system, a bicruciate substituting (BCS) knee implant system, or a posterior stabilized (PS) knee implant system.

27. A method of performing knee arthroplasty on a patient, comprising:
implanting a tibial component comprising on a prepared proximal end of a tibia of a knee joint of the patient, the tibial component comprising a base and a tibial post having an anterior side, the tibial post extending superiorly from the base; and
implanting a femoral component on a prepared distal end of a femur of the knee, the femoral component configured to engage the base of the tibial component and comprising:
a femoral box configured to receive the tibial post, the femoral box having a posterior side wall and an anterior side wall arranged opposite the posterior side wall,
wherein the anterior side wall is formed with an angled section angled anteriorly to allow for clearance between the anterior side of the tibial post and the anterior side wall during extension and flexion of a knee implanted with the knee implant system.

28-39. (canceled)
